(19) United States
(12) Patent Application Publication
(10) Pub. No.: US 2023/0380415 A1
(43) Pub. Date: Nov. 30, 2023

(54) LIQUID-TIGHT CASE FOR TISSUE DEVICE AND SYSTEM HAVING SAME

(71) Applicant: 3D BioLabs, LLC, Chadds Ford, PA (US)

(72) Inventor: Joseph Phillip Vacanti, Winchester, MA (US)

(21) Appl. No.: 18/306,132

(22) Filed: Apr. 24, 2023

Related U.S. Application Data
(60) Provisional application No. 63/333,905, filed on Apr. 22, 2022.
(60) Provisional application No. 63/495,046, filed on Apr. 7, 2023.

(51) Int. Cl. A01N 1/02

(52) U.S. Cl. CPC A01N 1/0247

(57) ABSTRACT

A liquid-tight case includes a housing to enclose a tissue device, which has a first channel network and a second channel network in fluidic communication with the first channel network. The liquid-tight case also includes a first inlet port, a second inlet port, and at least one outlet port. The first inlet port is formed at or coupled with the housing, and configured to be in fluidic communication with the first channel network of the tissue device. The second inlet port is formed at or coupled with the housing, and configured to be in fluidic communication with the second channel network of the tissue device. The at least one outlet port is formed at or coupled with the housing. The liquid-tight case allows the tissue device to have more flexible and more complex design with more suitable or modified materials for cell migration and tissue formation.

### CROSS-REFERENCE TO RELATED APPLICATION(S)

The present Application claims priority to U.S. Provisional Patent Application No. 63/333,905, entitled “Liquid-Tight Case for Tissue Device and System Having Same,” filed Apr. 22, 2022, which is hereby incorporated by reference in its entirety for all purposes. The present Application also claims priority to U.S. Provisional Patent Application No. 63/495,046, entitled “Liquid-Tight Case for Tissue Device and System Having Same,” filed Apr. 7, 2023, which is hereby incorporated by reference in its entirety for all purposes.

### TECHNICAL FIELD

The present disclosure generally relates to liquid-tight cases for enclosing tissue devices, and systems having tissue devices enclosed within liquid-tight cases, and controlling a flow of a medium therethrough.

### BACKGROUND

Existing tissue devices experience a number of issues, including leaking, cracking, and delaminating, and further issues with controlling a flow through microchannel device. For instance, conventional approaches for utilizing tissue devices have been susceptible to tearing or delamination when subjected to compressive forces that progressive leads to an undesirable collapsing structure.

Given the current state of the art, there remains a need for liquid-tight cases and systems that address the abovementioned issues.

The information disclosed in this Background section is provided for an understanding of the general background of the invention and is not an acknowledgement or suggestion that this information forms part of the prior art already known to a person skilled in the art.

### SUMMARY

In an exemplary embodiment, the present disclosure describes liquid-tight cases for enclosing and protecting tissue devices. In various embodiments, the present disclosure also provides systems having tissue devices enclosed in liquid-tight cases.

In various embodiments, the present disclosure provides a liquid-tight case including a housing, a first inlet port, a second inlet port, and at least one outlet port. The housing is configured to enclose a tissue device within. The tissue device includes a first channel network and a second channel network in fluidic communication with the first channel network. The first inlet port is coupled with the housing, and configured to be in fluidic communication with the first channel network of the tissue device. The second inlet port is coupled with the housing, and configured to be in fluidic communication with the second channel network of the tissue device. The at least one outlet port is coupled with the housing.

In various embodiments, the present disclosure provides a liquid-tight case including a housing, a first inlet port, a second inlet port and at least one outlet port. The housing is configured to enclose a tissue device within. The first inlet port is coupled with the housing, and configured to receive a first plurality of viable cells and pass the first plurality of viable cells to the tissue device. The second inlet port is coupled with the housing, and configured to receive a second plurality of viable cells and pass the second plurality of viable cells to the tissue device. The at least one outlet port is coupled with the housing.

In some embodiments, the tissue device includes a first channel network and a second channel network. The first channel network is in fluidic communication with the first inlet port of the liquid-tight case to receive the first plurality of viable cells. The second channel network is in fluidic communication with the second inlet port of the liquid-tight case to receive the second plurality of viable cells. The first and second channel networks are in fluidic communication with each other.

In some embodiments, the tissue device is a device simulating arterial blood flow.

In some embodiments, the tissue device is a liver device.

In some embodiments, the at least one outlet port includes a first outlet port, in which a first portion of the first outlet port is in fluidic communication with a second portion of the first inlet port, thereby providing fluidic communication through the tissue device.

In some embodiments, the housing includes a base, a lid, and a seal. The base is formed with a hollow space configured to house the tissue device. The lid is coupled with the base to enclose the tissue device within the hollow space. The seal is disposed between the base and the lid to enhance sealing of the liquid-tight case.

In some embodiments, the lid is coupled with the base by one or more fasteners, snap-fitting, press-fitting, one or more adhesives, a tape, or a combination thereof.

In some embodiments, the seal is made of an elastomeric material.

In some embodiments, the seal is an O-ring.

In some embodiments, the base includes a rim and a groove formed along the rim to accommodate the seal.

In some embodiments, the first inlet port, second inlet port and at least one outlet port are coupled with the lid or base.

In some embodiments, the first inlet port, the second inlet port, or each of the first and second inlet ports includes a barbed outer connector or coupled with an exterior side of the lid or base to facilitate tubing.

In some embodiments, the at least one outlet port includes a first outlet port. The base of the housing is monolithically formed with a sheath configured to allow tubing of the first outlet port to pass through the housing and to be connected directly to a portion of the tissue device.

In some embodiments, the first and second inlet ports are or coupled with a first portion of the base and the at least one outlet port is or coupled with a second portion of the base.

In some embodiments, each of the first and second inlet ports includes a barbed outer connector formed at an exterior side of the first portion of the base to facilitate tubing. Additionally, or optionally, a barbed inner connector is formed at an interior side of the first portion of the base to facilitate tubing and direct connection to the tissue device.

In some embodiments, the first and second portions of the base are opposite to each other.

In some embodiments, the first and second inlet ports are coupled with the lid.

In some embodiments, each of the first and second inlet ports includes a barbed outer connector is coupled with an exterior side of the lid to facilitate tubing. Additionally, or optionally, in some embodiments, a barbed inner connector is formed at or coupled with an interior side of the lid to facilitate tubing and direct connection to the tissue device.

In various embodiments, the present disclosure provides a system including a liquid-tight case disclosed herein, and a tissue device enclosed within the liquid-tight case and in fluidic communication with the first inlet port, the second inlet port and the at least one outlet port.

In some embodiments, the system is implantable to a body of a human subject or an animal.

In some embodiments, the animal is a rat or a pig.

In some embodiments, the system is one of cardiac vascularized left ventricle muscle patches, face transplants, limbs, digits, and kidneys.

In various embodiments, the provides an apparatus for controlling a flow through a liquid-tight case and/or a tissue device accommodated by the liquid-tight case. In some embodiments, the apparatus includes a plurality of pumps. In some embodiments, the apparatus includes a manifold interconnecting the plurality of pumps. In some embodiments, the apparatus includes an outlet tubing coupled to the manifold.

The liquid-tight cases and systems of the present disclosure have other features and advantages that will be apparent from, or are set forth in more detail in, the accompanying drawings, which are incorporated herein, and the following Detailed Description, which together serve to explain certain principles of exemplary embodiments of the present disclosure

### BRIEF DESCRIPTION OF THE DRAWINGS

The foregoing summary, as well as the following detailed description of embodiments of the systems and devices, will be better understood when read in conjunction with the appended drawings of exemplary embodiments. It should be understood, however, that the invention is not limited to the precise arrangements and instrumentalities shown.

In the drawings:

FIG. 1 is a perspective view illustrating schematically an exemplary system in accordance with some exemplary embodiments of the present invention;

FIG. 2 is a partially-cutout side view illustrating schematically the system of FIG. 1 in accordance with some exemplary embodiments of the present invention;

FIG. 3 is an exploded view illustrating schematically the system of FIG. 1 in accordance with some exemplary embodiments of the present invention;

FIG. 4 is a perspective view illustrating schematically an exemplary system in accordance with some exemplary embodiments of the present invention;

FIG. 5 is a partially-cutout top view illustrating schematically the system of FIG. 4 in accordance with some exemplary embodiments of the present invention;

FIG. 6 is an exploded view illustrating schematically the system of FIG. 4 in accordance with some exemplary embodiments of the present invention;

FIG. 7 is a top view illustrating schematically a variation of the system of FIG. 4 in accordance with some exemplary embodiments of the present invention;

FIG. 8 is a perspective view illustrating schematically an exemplary system in accordance with some exemplary embodiments of the present invention;

FIG. 9 is a side view illustrating schematically the system of FIG. 8 in accordance with some exemplary embodiments of the present invention;

FIG. 10A is a partially-cutout side view illustrating schematically the system of FIG. 8 in accordance with some exemplary embodiments of the present invention;

FIG. 10B is an enlarged view of a portion of FIG. 10A;

FIG. 11 is an exploded view illustrating schematically the system of FIG. 8 in accordance with some exemplary embodiments of the present invention;

FIG. 12 is a side view illustrating schematically the system of FIG. 8 in accordance with some exemplary embodiments of the present invention;

FIG. 13 is a partially-cutout side view illustrating schematically the system of FIG. 8 in accordance with some exemplary embodiments of the present invention;

FIG. 14 is a perspective view illustrating schematically a lip in accordance with some exemplary embodiments of the present invention;

FIG. 15 is another perspective view illustrating schematically the lip of FIG. 14 in accordance with some exemplary embodiments of the present invention;

FIG. 16 illustrates a perfusion test in accordance with some exemplary embodiments of the present invention;

FIG. 17 illustrates an apparatus for controlling a flow through a liquid-tight case and/or a tissue device accommodated by the liquid-tight case in accordance with some exemplary embodiments of the present invention;

FIG. 18 illustrates an apparatus for controlling a flow through a liquid-tight case and/or a tissue device accommodated by the liquid-tight case in accordance with some exemplary embodiments of the present invention;

FIG. 19 illustrates a first portion of an apparatus for controlling a flow through a liquid-tight case and/or a tissue device accommodated by the liquid-tight case in accordance with some exemplary embodiments of the present invention;

FIG. 20 illustrates a second portion of an apparatus for controlling a flow through a liquid-tight case and/or a tissue device accommodated by the liquid-tight case in accordance with some exemplary embodiments of the present invention;

FIG. 21 illustrates a manifold of an apparatus for controlling a flow through a liquid-tight case and/or a tissue device accommodated by the liquid-tight case in accordance with some exemplary embodiments of the present invention;

FIG. 22 illustrates an apparatus for controlling a flow through a liquid-tight case and/or a tissue device accommodated by the liquid-tight case in accordance with some exemplary embodiments of the present invention;

FIG. 23 illustrates a side view of an apparatus for controlling a flow through a liquid-tight case and/or a tissue device accommodated by the liquid-tight case in accordance with some exemplary embodiments of the present invention;

FIG. 24 illustrates a second side view of the apparatus of FIG. 23;

FIG. 25 illustrates a cross-sectional view of an apparatus for controlling a flow through a liquid-tight case and/or a tissue device accommodated by the liquid-tight case in accordance with some exemplary embodiments of the present invention;

FIG. 26 illustrates a chart depicting various dimensions for an apparatus for controlling a flow through a liquid-tight case and/or a tissue device accommodated by the liquid-tight case in accordance with some exemplary embodiments of the present invention; and

FIG. 27 illustrates a system including a liquid-tight case and an apparatus for controlling a flow through a liquid-tight case and/or a tissue device accommodated by the liquid-tight case in accordance with some exemplary embodiments of the present invention.

### DETAILED DESCRIPTION

Disclosed herein are systems and apparatuses including liquid-tight cases for enclosing and protecting tissue devices. An exemplary liquid-tight case of the present invention generally includes a housing configured to enclose a tissue device within, and one or more ports configured to connect the tissue device with one or more component(s) (e.g., other organs, devices, structures, or sources) outside of the liquid-tight case, such as to provide nutrition, viable cells, other materials, or a combination thereof to the tissue device. An exemplary liquid-tight case of the present invention can have any shape and any size, allowing for the liquid tight case to accommodate a tissue devices of varying size.

Exemplary liquid-tight cases of the present invention solve a number of issues in existing tissue devices including leaking, cracking, and delaminating. In some embodiments, the liquid-tight cases of the present disclosure allow tissue devices to have a flexible and/pr a complex (e.g., fractal) design. For instance, with the protection of exemplary liquid-tight cases of the present invention, tissue devices can be configured to achieve a variety of functions with more suitable shapes, structures, and internal channels. Incorporating exemplary liquid tight cases of the invention into various systems, tissue devices can also be made of more suitable or modified materials (e.g., hydrogel) for cell migration and tissue formation. With exemplary liquid-tight cases of the present invention, it is possible to construct a variety of systems (a system refers to a tissue device enclosed in a liquid-tight case of the present invention), such as cardiac vascularized left ventricle muscle patches, face transplants, limbs, digits, and kidneys.

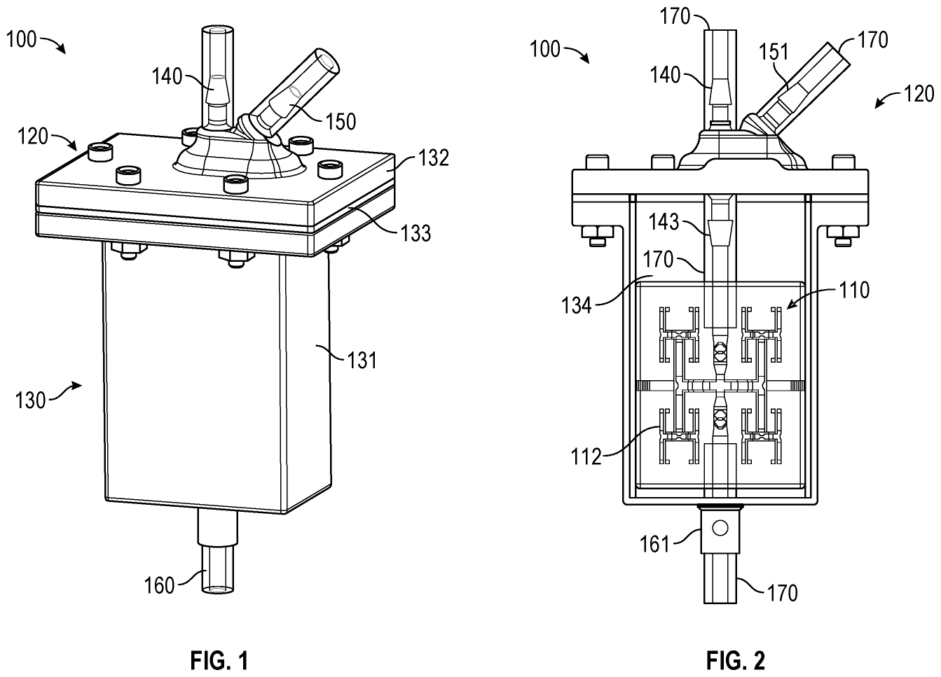

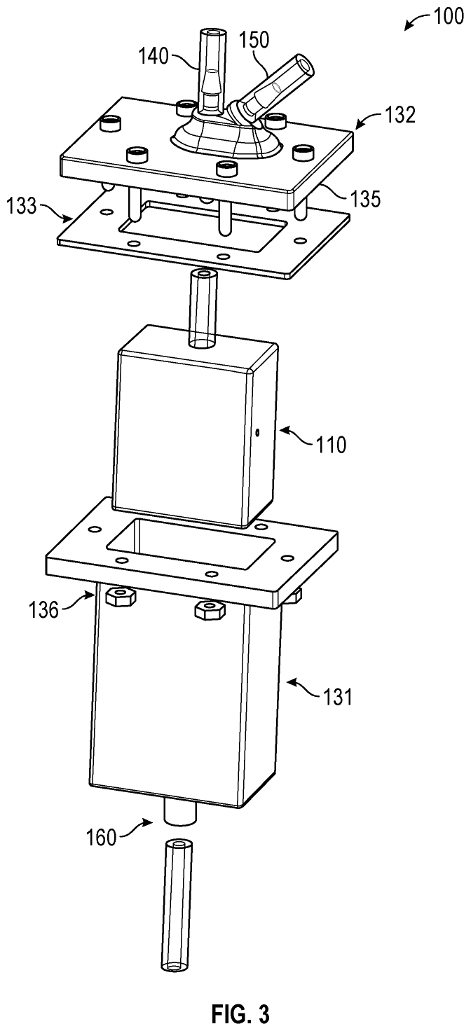

Referring now to the drawings in detail, wherein like reference numerals indicate like elements throughout, there is illustrated in FIGS. 1-3, an exemplary system 100 in accordance with some exemplary embodiments of the present invention. System 100 generally includes a tissue device, such as tissue device 110, and a liquid-tight case, such as liquid-tight case 120. The exemplary liquid-tight case is configured to protect the tissue device and ensure the tissue device to receive nutrition, viable cells and/or other materials. An exemplary tissue device can be printed, for example, using an additive manufacture method (e.g., at high resolution), which allows for forming the tissue device as a three-dimensional monolithic structure, at least in part. In some embodiments, the additive manufacturing method is selected from the group consisting of: binder jetting, material extrusion, material jetting, polyjet, powder bed, sheet lamination, and vat photopolymerization. In some embodiments, the additive manufacturing method of vat photopolymerization includes stereolithography (e.g., projection stereolithography). In some such embodiments, by using the additive manufacture method, a direction of each layer of the liquid tight case is formed in a first direction that is perpendicular to a direction of force applied to the liquid tight case. For instance, in some embodiments, the layer direction is not normal to a pull direction of the liquid tight case. However, the present disclosure is not limited thereto.

For instance, in some embodiments, the liquid-tight case includes a polymer that is selected, at least in part, based on a degree of swelling of the polymer. In some embodiments, the degree of swelling is based on or more parameters including one or more dimensional parameters of the liquid tight case (e.g., planar swelling, volumetric swelling), one or more mass parameters of the liquid tight case (e.g., mass swelling), a temporal parameter of the liquid tight case (e.g., per unit time), or a combination thereof. In some embodiments, the degree of swelling is a ratio of a difference between a wet weight and a dry weight in comparison to the wet weight of the liquid tight case. For instance, in some embodiments, the swelling degree is determined by: Swelling Ratio=Wwet−WdryWwet.

In some embodiments, the liquid-tight case includes a polymer selected, at least in part, based on a porosity in order to allow a candidate subject cell to attach to a pore of the liquid-tight case (e.g., a pore of the tissue device) without seeping through to an environment, thereby limiting a permeability of the liquid-tight case. In some embodiments, a median size of a pore of the liquid-tight case is from about 5 microns (μm) to about 300 μm, from about 19 μm to about 231 μm, from about 25 μm to about 175 μm, or from about 60 μm to about 100 μm. In some embodiments, a median size of a pore of the liquid-tight case is at least 5 μm, at least 19 μm, at least 25 μm, at least 60 μm, at least 100 μm, at least 175 μm, at least 231 μm, or at least 300. In some embodiments, a median size of a pore of the liquid-tight case is at most 5 μm, at most 19 μm, at most 25 μm, at most 60 μm, at most 100 μm, at most 175 μm, at most 231 μm, or at most 300.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to ±5%.

In some embodiments, the polymer material of the liquid-tight case includes a member selected from poly-dimethyl-siloxane (PDMS), poly-glycerol-sebacate (PGS), polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyesterspolyacrylates, polymethacrylate, acyl substituted cellulose acetates, non-degradable polyurethanes, polystyrenes, polyvinyl chloride, polyvinyl flouride, polyvinyl imidazole, chlorosulphonated polyolifins, polyethylene oxide, polyvinyl alcohol, Teflon©, nylon silicon, shape memory materials, e.g., poly(styrene-block-butadiene), polynorbornene, hydrogels, metallic alloys, oligo(ε-caprolactone)diol as switching segment/oligo(p-dioxyanone)diol as physical crosslink, and a combination thereof. For instance, in some embodiments, the case utilizes a PLGA polymer which is a PLA/PGA polymer having a ration of 50/50. An exemplary PLA/PGA polymer has a molecular weight of about 30,000 Da. However, the present disclosure is not limited thereto.

Liquid-tight case 120 generally includes a housing, such as housing 130, configured to enclose a tissue device within. For instance, in some embodiments, housing 130 includes a base 131, a lid 132, and a seal 133. In some embodiments, the base, the lid, the seal, or a combination thereof is made by molding process, a printing process (e.g., additive manufacture method), or the like. In some embodiments, the base is formed with a hollow space 134 configured to house (e.g., accommodate) the tissue device. In some embodiments, the lid is coupled with the base to enclose the tissue device within the hollow space. For instance, in some embodiments, the base has an opening on the top and the lip is coupled with the top of the base (e.g., by coupling with the opening of the base)). In some embodiments, the housing, the base, the lid, the seal, or a combination thereof is formed as three-dimensional monolithic device, which improves a material integrity and durability of the liquid-tight case. For instance, in some embodiments, the housing, the base, the lid, the seal, or the combination thereof is integrally formed with the other of the housing, the base, the lid, the seal, or the combination thereof. However, the present disclosure is not limited thereto.

In some embodiments, the coupling of the lid and base is achieved by any suitable means, including but not limited to screws, bolts, pins, rivets, adhesives, snap-fitting, press-fitting, tapes, or the like. For instance, as a non-limiting example, in some embodiments, the lid is fastened to the base by a plurality of fasteners such as one or more bolts 135 and/or one or more nuts 136. While 6 pairs of bolts and nuts are illustrated, it should be noted that any number of fasteners (e.g., 2, 3, 4, 5, 6, 7, 8, or more than 8) can be used. Also, in some embodiments, the fasteners are spaced apart from each other evenly or non-evenly.

In some embodiments, the seal is disposed between the base and the lid to enhance sealing of the liquid-tight case. In some embodiments, the seal is made of an elastomeric material, for instance, by molding, printing or the like. For instance, in some embodiments, the seal is a mechanical seal that inhibits, retards, terminates, etc. fluidic communication between an interior volume and an environment. For instance, in some embodiments, the seal sits between the lid and the base and is compressed by a fastener mechanism, such as by tightening the bolts, thereby creating a liquid-tight (e.g., water-tight or waterproof) seal from the compression of seal interposing between the lid and the base. Accordingly, in some such embodiments, the seal prevents a flow of medium between a first surface of the base and a second surface of the lid. Moreover, in some such embodiments, by compressing the seal between first surface of the base and a second surface of the lid, the seal has an improved fit therebetween to maintain retarding the flow therebetween independent of type of medium, pressure, or temperature of the liquid-tight case. In some embodiments, the lid and/or the base includes a groove that is configured to accommodate the seal. In some embodiments, a first width of the groove is greater than a second width of the seal in an uncompressed state and less than a third width of the seal in a compressed state. However, the present disclosure is not limited thereto.

In some embodiments, a fastener is utilized to couple the lid and the base of the housing. For instance, in some embodiments, the fastener includes an adhesive layer (e.g., medical and/or surgical tape) that is used to encompass (e.g., wrap around) the fasteners or the like to reduce sharp edges and corners of the liquid-tight case. Alternatively, in another embodiment, the lid and base are formed with countersinks so that the nuts and bolts are inserted flush with the surface of the lid or base. In a further embodiment, nuts are countersunk in a hole or cut with a shape that would simplify fastening (e.g., no wrench needed if a nut countersunk in a snug hexagonal hole or the like). In some embodiments, the liquid-tight case includes one or more rounded or chamfer edges, which provide for a smooth exterior for the liquid-tight case. However, the present disclosure is not limited thereto. In some embodiments, the liquid-tight case includes

In some embodiments, the fastener is flush with a surface of the housing, such that an end portion of the fastener in in plane with an end portion of the housing. Thus, the fastener does not substantially extend past the end portion of the housing. However, the present disclosure is not limited thereto. For instance, in some embodiments, the fastener is recessed from the surface of the housing and/or protrudes from the surface of the housing. In some embodiments, the fastener and the housing are flush. The term “flush,” as used herein, is defined as a surface of a first component and a same respective surface of a second component to have a distance or level separating the first component and the second component to be 0.0 cm, within a tolerance of 50 μm, within a tolerance of 0.1 mm, within a tolerance of 0.1 cm, or within a tolerance of 0.25 cm. In some embodiments, the same respective surface of the second component is coplanar to the surface of the first component. For instance, in some embodiments, the lid is considered to be flush with the base can be either internally disposed within a portion of the base or integrated with the base. However, the present disclosure is not limited thereto. Accordingly, in some embodiments, the housing includes one or more rounded edge portions, which allows for easier insertion, such as if the liquid-tight case is implanted in a subject.

In some embodiments, the housing is configured to minimize free space (e.g., one or more voids) within an interior of the liquid-tight case. For instance, in some embodiments, the housing is configured to accommodate the tissue device without decaying some or all of the cells accommodated by the tissue device. In some embodiments, the housing is configured to maximize an active area of the tissue device. However, the present disclosure is not limited thereto.

Liquid-tight case 120 also includes one or more ports to connect the tissue device with one or more components (e.g., organs, other devices, sources, or the like) outside of the liquid-tight case, such as to provide nutrition, viable cells, other materials, or a combination thereof to the tissue device. The one or more ports are formed at or coupled with the housing. For instance, in some embodiments, the liquid-tight case includes first inlet port 140, second inlet port 150, and at least one outlet port. In some embodiments, the one or more ports are integrally formed with the housing, such as by forming a three-dimensional monolithic structure. In some embodiments, the at least one outlet port includes first outlet port 160. In some embodiments, the at least one outlet port includes one or more additional or optional outlet ports in addition to the first outlet port. In some embodiments, the at least one outlet port includes one outlet port, two outlet ports, three outlet ports, or four outlet ports. In some embodiments, the at least one outlet port includes no more than two outlet ports, no more than three outlet ports, or no more than four outlet ports. In some embodiments, the at least one outlet port includes at least two outlet ports, at least three outlet ports, or at least four outlet ports. However, the present disclosure is not limited thereto.

In some embodiments, the first inlet port, second inlet port and at least one outlet port have the same, similar or different size, shape, material, structure, or the like. Each of the first inlet port, second inlet port and at least one outlet port can be formed at or coupled with the lid or base, independently, separately or jointly with others. For instance, as a non-limiting example, the first inlet port and second inlet port are formed at or coupled with the lid, with the first outlet port formed at or coupled with the base. In some embodiments, each port is configured to exhibit laminar flow therein. In some embodiments, each port is dimensioned in accordance with Murray's law.

In some embodiments, the base is formed monolithically (e.g., as a three-dimensional monolithic structure) with a sheath, such as sheath 161. The sheath is configured to allow a tube, generally designated by reference numeral 170, to pass and to be connected directly to the tissue device (e.g., connected directly to an output of the first channel network of the tissue device), for instance, by adhesive(s) or the like. Examples of the adhesive include but not limited to silicone, cyanoacetate, styrene butadiene copolymer or the like. In some such embodiments, the tube passing though the sheath and connected to the tissue device serves as the first outlet port. The sheath with tubing can be wrapped around by tape(s) or glued by adhesive(s) to provide an additional seal.

In some embodiments, the first inlet port includes a barbed outer connector (or barb), such as barbed outer connector 141, formed at or coupled with an exterior side of the lid. Barbed outer connector 141 is configured to facilitate tubing, e.g., to enable easy and/or tight connection of a tube. Similarly, in some embodiments, the second inlet port includes a barbed outer connector, such as barbed outer connector 151, formed at or coupled with an exterior side of the lid to facilitate tubing. In some embodiments, a barbed inner connector, such as barbed inner connector 143, is formed at or coupled with an interior side of the lid to facilitate tubing. In some embodiments, the tube connected with barbed inner connector 143 is connected directly to the tissue device (e.g., connected directly to an input of the first channel network of the tissue device), for instance, by adhesive(s) or the like.

In some embodiments, the barbed connectors for different ports are configured the same or differently, e.g., having the same shape or different shapes, having the same size or different sizes, or the like. Similarly, the tubing, while generally designated by reference numeral 170, can have the same configuration or different configurations for different barbed connectors and different ports. For instance, in some embodiments, all barbed outer connectors (or barbs) are configured substantially the same, and the tubing for different barbed outer connectors and/or the first outlet port is the same. In some embodiments, all tubing barbs are designed for tubing with an inner diameter (ID) of about ⅛ of an inch and an outer diameter (OD) of about ¼ of an inch. In some embodiments, one or more barbs are designed for tubing with ID less than ⅛ of an inch and/or OD less than ¼ of an inch. In some embodiments, one or more barbs are designed for tubing with ID greater than ⅛ of an inch and/or OD greater than ¼ of an inch.

In some embodiments, the 3-barb Y-joint (i.e., barbed outer connector 141, barbed outer connector 151 and barded inner connector 143) are formed monolithically with the lid, for instance by molding or printing or the like. In an alternative embodiment, the 3-barb Y-joint is made, for instance by molding or printing or the like, as a separate part and coupled with the lip. A separate 3-barb Y-joint can be made of a more durable plastic. In a further alternative embodiment, the first or second inlet port is constructed in a way similar to first outlet port 160 with a sheath rather than a barb.

In some embodiments, the first inlet port is configured to receive a first plurality of viable cells and pass the first plurality of viable cells to the tissue device. The second inlet port is configured to receive a second plurality of viable cells and pass the second plurality of viable cells to the tissue device. For instance, in some embodiments, the first inlet port is a portal vein (PV) port that receives a blood flow and passes it to the first channel network of the tissue device. The second inlet port is a hepatobiliary (HB) port that provides an inflow and/or outflow of bile and is in fluidic communication with the second channel network of the tissue device. The first outlet port is a PV outlet port, and in in fluidic communication with the first inlet port through the tissue device. The first outlet port allows the blood flow to exit after it is distributed across the tissue device to enable oxygen transport and cell nourishment of both networks.

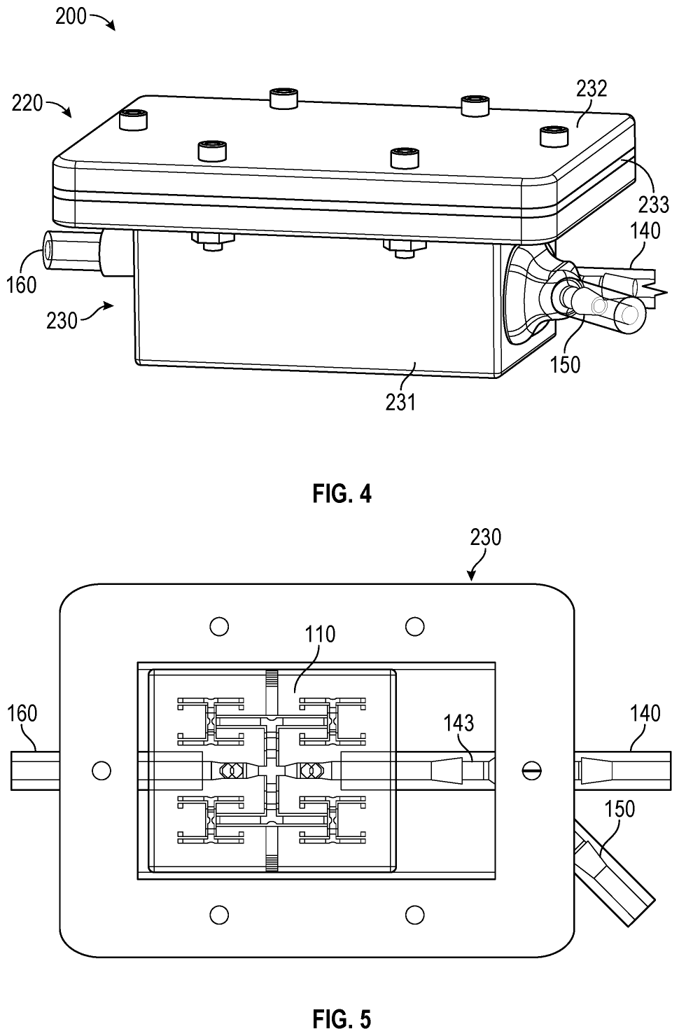

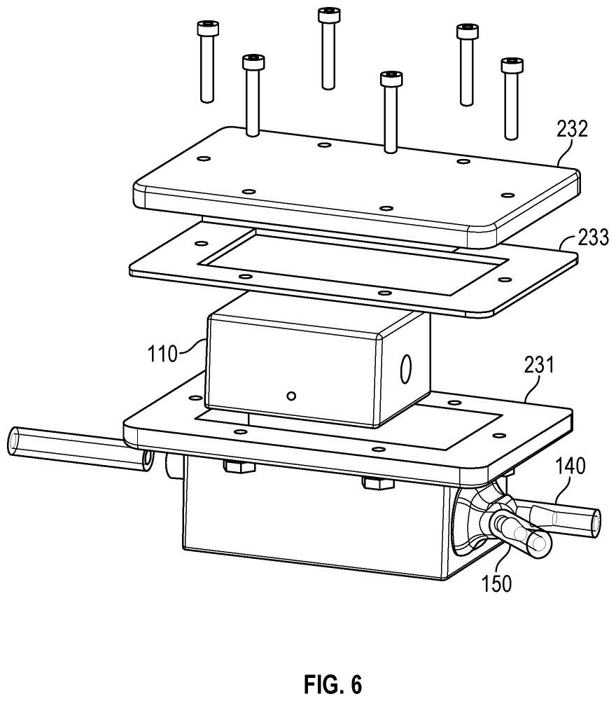

Referring to FIGS. 4-6, there is illustrated an exemplary system 200 in accordance with some exemplary embodiments of the present invention. System 200 generally includes a tissue device, such as tissue device 110, and a liquid-tight case, such as liquid-tight case 220, to protect the tissue device and ensure the tissue device to receive nutrition, viable cells and/or other materials. Liquid-tight case 220 is similar to liquid-tight case 120 except in liquid-tight case 220, (i) the base has an opening on a side rather than on a top, (ii) the lip is coupled with the side of the base, and (iii) all of the first inlet port, second inlet port and first outlet port are formed at or coupled with the base.

For instance, in some embodiments, liquid-tight case 220 include housing 230 configured to enclose the tissue device within. Housing 230 includes a base 231, a lid 232, and a seal 233. The base, lid and/or seal can be made by molding, printing or the like. The base has an opening on a side and the lip is coupled with the side of the base.

In some embodiments, liquid-tight case 220 also includes first inlet port 140, second inlet port 150 and first outlet port 160. The first inlet port and second inlet port are formed at or coupled with a first portion of base 231. Similar to liquid-tight case 120, in some embodiments, the first inlet port, the second inlet port or each of the first and second inlet ports of liquid-tight case 220 includes a barbed outer connector formed at an exterior side of the first portion of the base to facilitate tubing. In some embodiments, a barbed inner connector, such as bared inner connector 143, is also formed at an interior side of the first portion of the base to facilitate tubing and direct connection to the tissue device. However, the present disclosure is not limited thereto.

In some embodiments, the first outlet port is formed at or coupled with a second portion of base 231. Similar to liquid-tight case 120, in some embodiments, base 231 is formed monolithically with a sheath, such as sheath 161, at the second portion to allow a tube to pass and to be connected directly to the tissue device. In some embodiments, the tube passing though the sheath and connected to the tissue device serves as the first outlet port. The sheath with tubing can be wrapped around by tape(s) or glued by adhesive(s) to provide an additional seal.

In some embodiments, the first and second portions of base 231 are opposite to each other. For instance, in some embodiments, the first portion of base 231 is the wall of the base on the right hand side of the figure and the second portion of base 231 is the wall of the base on the left hand side of the figure.

Similar to liquid-tight case 120, in some embodiments, the 3-barb Y-joint of liquid-tight case 220 are formed monolithically with the first portion of base 231, for instance by molding or printing or the like. In an alternative embodiment, the 3-barb Y-joint is made, for instance by molding or printing or the like, as a separate part and coupled with the first portion of base 231. In a further alternative embodiment, the first or second inlet port is constructed in a way similar to first outlet port 160 with a sheath rather than a barb.

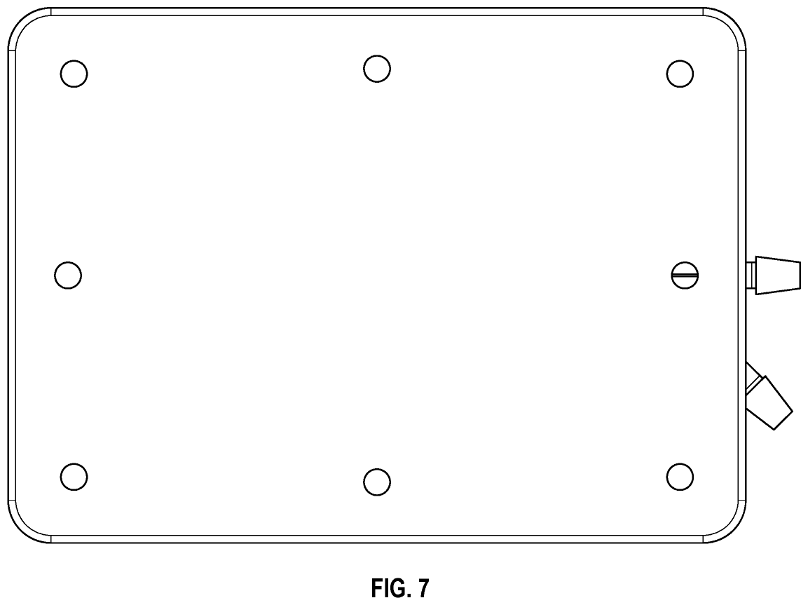

In some embodiments, similar to liquid-tight case 120, the lid is fastened to the base, for instance, by bolts and nuts. The lid can be fastened to the base by any number of fasteners (e.g., any number of pairs of bolts and nuts), such as 2, 3, 4, 5, 6, 7, 8, more than 8, more than 10, more than 15, or more than 20, depending on the size and shape of the lid and base. As a non-limiting example, FIGS. 4-6 illustrate the lid fastened to the base by 6 fasteners. As another non-limiting example, FIG. 7 illustrates the lid to be fastened to the base by 8 fasteners, which is indicated by the additional bolt holes shown in the figure.

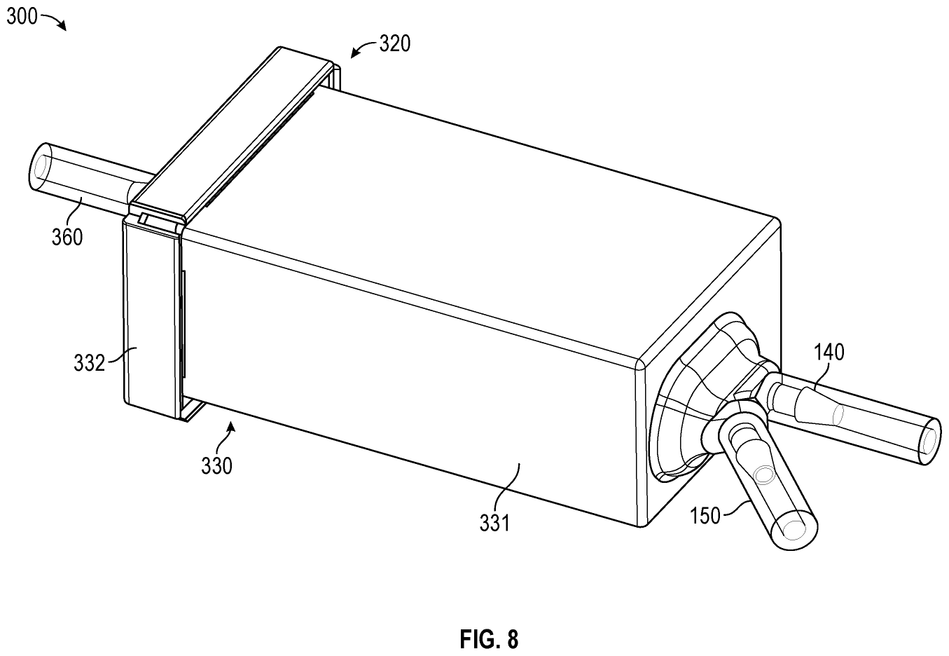

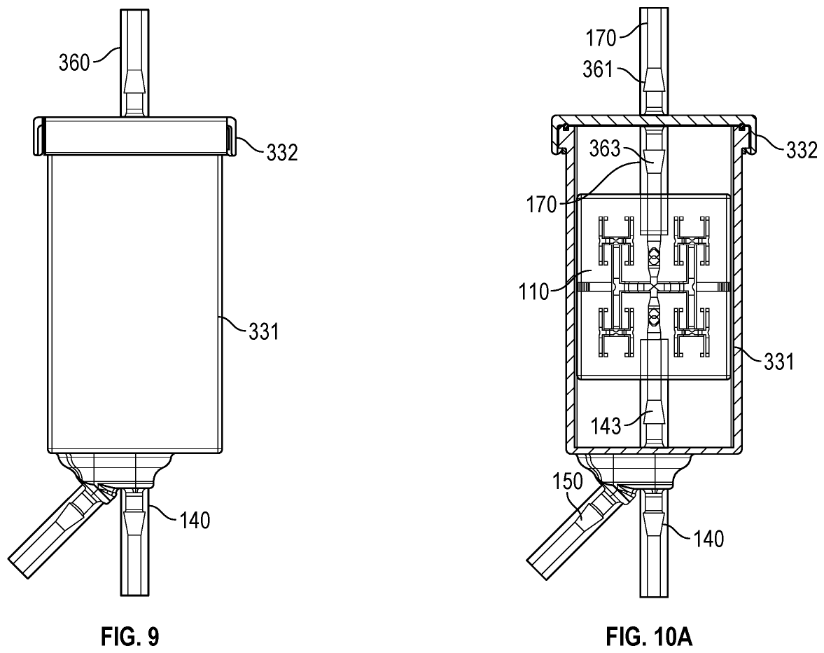

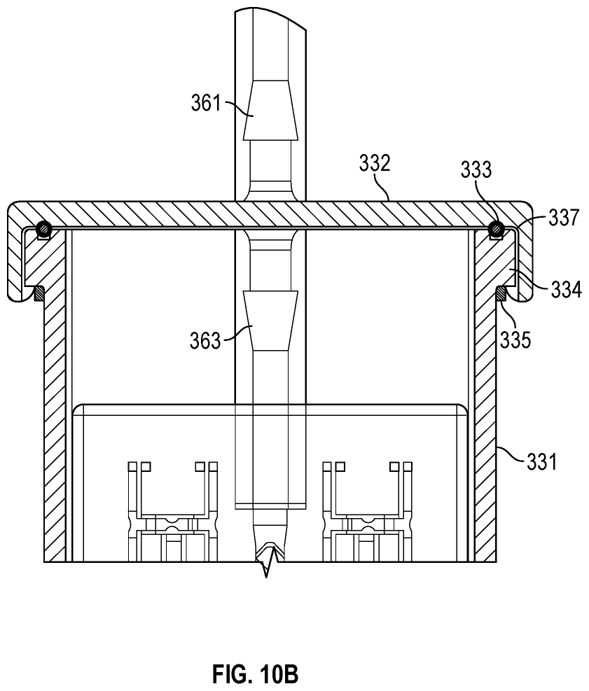

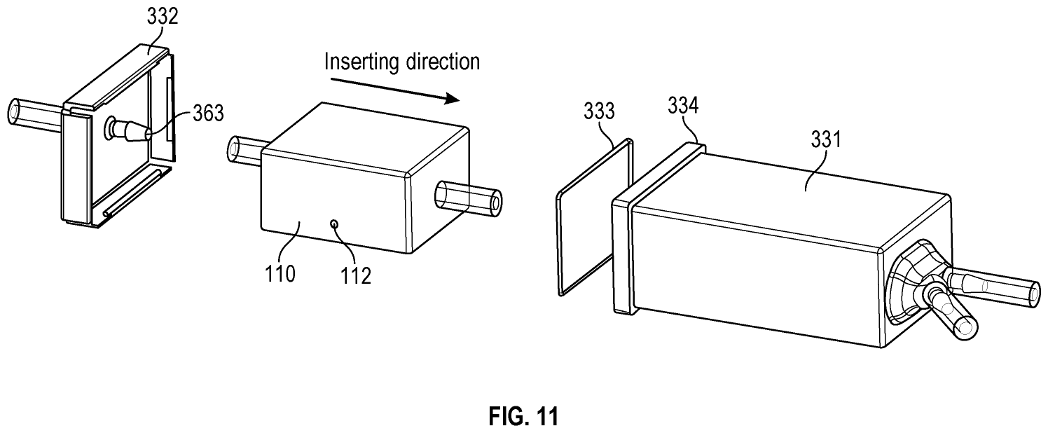

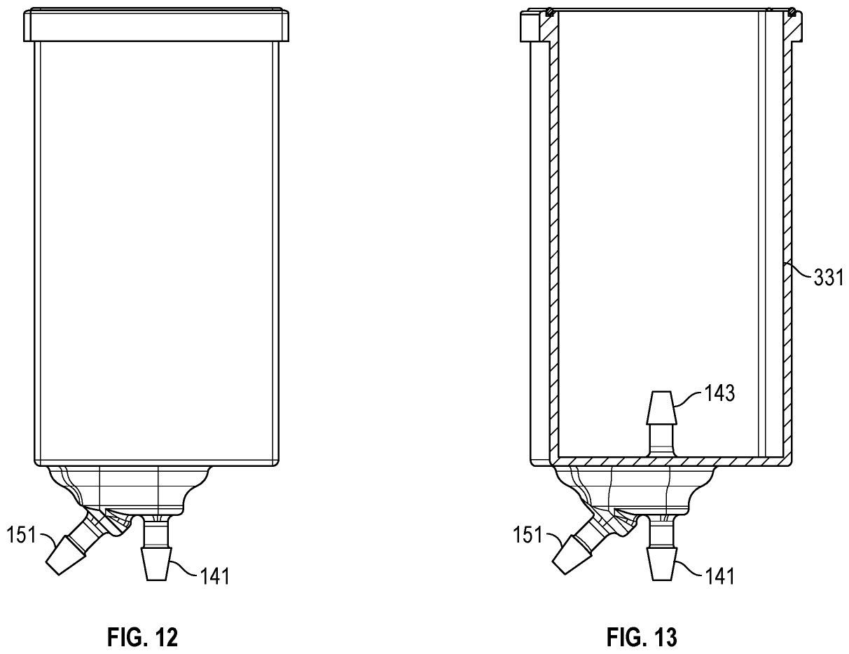

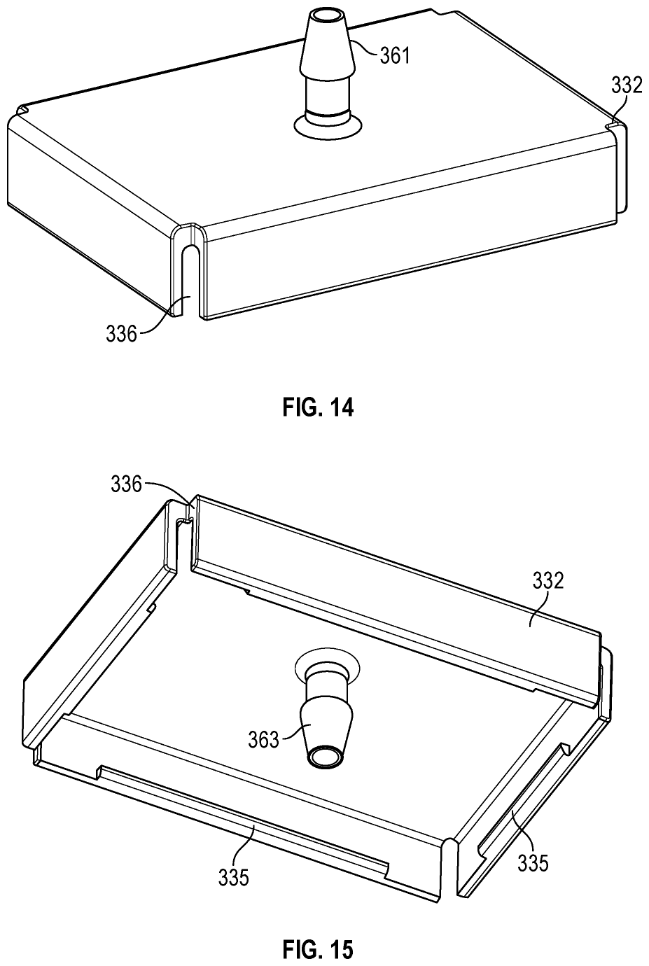

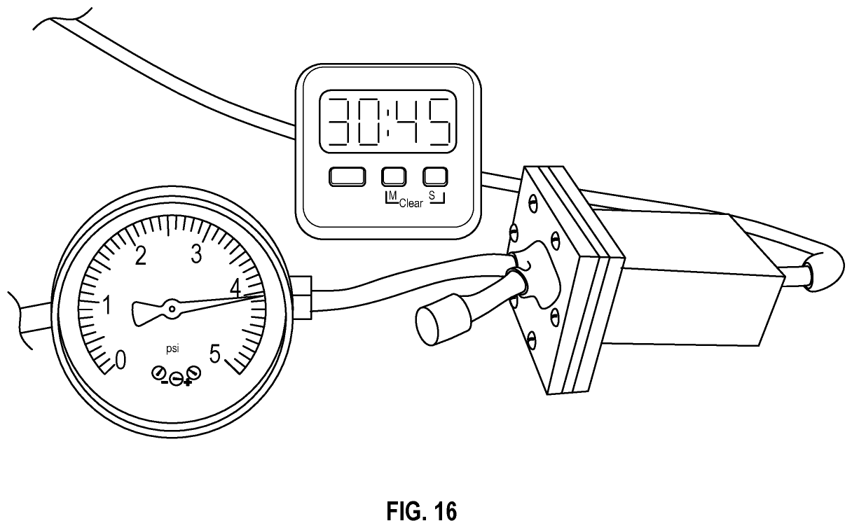

Referring to FIGS. 8-15, there is shown an exemplary system 300 in accordance with some exemplary embodiments of the present invention. System 300 generally includes a tissue device, such as tissue device 110, and a liquid-tight case, such as liquid-tight case 320, to protect the tissue device and ensure the tissue device to receive nutrition, viable cells and/or other materials. Liquid-tight case 320 is similar to liquid-tight case 120 except (i) the lip of liquid-tight case 320 is fastened to the base not by nuts and bolts but rather by snap-fitting, press-fitting or the like, (ii) the first inlet port and second inlet port of liquid-tight case 320 are formed at or coupled with the base, and (iii) the first outlet port is formed at or coupled with the lid.

For instance, in some embodiments, liquid-tight case 320 include housing 330 configured to enclose the tissue device within. Housing 330 includes a base 331, a lid 332, and a seal 333. The base, lid and/or seal can be made by molding, printing or the like. The base has an opening on the top, and is formed with a rim, such as rim 334. The lid is formed with one or more snaps, such as snap 335, on at least one side of the lid to fasten to the rim on the base. In some embodiments, the lid is formed with one or more snaps on all sides of the lid to fasten to the rim of the base. In another embodiment, a snap is formed on each side of the lid. In still another embodiment, at least one side of the lid is formed with more than one snap. In some embodiments, for snapping flexibility of an otherwise stiff material, the lid is formed with one or more cutouts, such as cutout 336, in one or more corners of the lid. In some embodiments, the lid is formed with a cutout in each of the corners.

In some embodiments, a groove, such as groove 337, is formed along the rim to accommodate seal 333. In some embodiments, seal 333 is an O-ring made by printing. The seal can be placed in the groove of the base, glued in place or the like.

In some embodiments, liquid-tight case 320 also includes first inlet port 140, second inlet port 150 and at least one outlet port such as first outlet port 360. The first inlet port and second inlet port are formed at or coupled with a portion of base 331, e.g., the bottom of the base. Similar to liquid-tight case 120, in some embodiments, the first inlet port, the second inlet port or each of the first and second inlet ports of liquid-tight case 320 includes a barbed outer connector formed at an exterior side of the portion of the base to facilitate tubing. In some embodiments, a barbed inner connector, such as bared inner connector 143, is also formed at an interior side of the portion of the base to facilitate tubing and direct connection to the tissue device.

In some embodiments, first outlet port 360 is formed at or coupled with lid 332. Similar to first inlet port 140, in some embodiments, first outlet port 360 includes a barbed outer connector, such as barbed outer connector 361, formed at an exterior side of lid 332 to facilitate tubing. In some embodiments, a barbed inner connector, such as bared inner connector 363, is also formed at an interior side of lid 332 to facilitate tubing and direct connection to the tissue device. The barbed inner and outer connectors isolate external tubing motion from the components inside the liquid-tight case.

In some embodiments, similar to liquid-tight case 120, in some embodiments, the 3-barb Y-joint of liquid-tight case 320 are formed monolithically with the portion (e.g., the bottom) of base 331, for instance by molding or printing or the like. In an alternative embodiment, the 3-barb Y-joint is made, for instance by molding or printing or the like, as a separate part and coupled with the portion of base 331. In a further alternative embodiment, the first or second inlet port is constructed in a way similar to first outlet port 160 with a sheath rather than a barb.

In some embodiments, while the liquid-tight cases (e.g., case 120, case 220, case 320) are illustrated to be of a substantially rectangular, or cubic, shape, it should be noted that these are non-limiting examples. The liquid-tight cases can have any suitable shapes and sizes depending on the applications and/or the shape/size/function of the tissue device. For instance, the base (e.g., base 131, base 231, base 331) can have a substantially cubic, cylindrical, spheric, dome shape or the like. Similarly, the lid (e.g., lid 132, lid 232, lid 332) and the seal (e.g., seal 133, seal 233, seal 333) can have a substantially rectangular, circular shape or the like to couple with the base. In some embodiments, the base includes a first polygonal shape and the lid includes a second polygonal shape different than the first polygonal shape.

Accordingly, the system (e.g., system 100, system 200, system 300) can have any suitable shapes and sizes depending on the applications and/or the shape/size/function of the tissue device. The system can be small, for instance, configured for implanting into a small animal such as a rat. The system can be large, for instance, configured for implanting into a large animal such as a pig. Other non-limiting examples of the system include but are not limited to cardiac vascularized left ventricle muscle patches, ears, bones, face transplants, limbs, digits, and kidneys.

Referring to FIG. 16, there is illustrated a perfusion test. The perfusion test is conducted to evaluate any leakage through an assembled system and to confirm secure connection between tubes and ports. While system 100 is shown in the figure, it should be noted that the perfusion test can be formed on system 200, system 300 or similar systems. Before the perfusion test, the printing resolution of internal channels of the tissue device is evaluated, for instance by microscopy. After confirming that all channels of the tissue device are open and perfusable, a flow (e.g., 100 ml of 1x PBS) is perfused through the assembled system for a period of time (e.g., an hour) at a certain flow rate (e.g., a flow rate of 192 ml/min) and the leakage rate is evaluated.

Accordingly, in some embodiments, the tissue device includes a sponge or a mesh that facilitates the culturing of cells. For instance, in some embodiments, the sponge of the insert is a Type 1 collagen sponge, such as that prepared from a dermis of a pig. In some embodiments, the tissue device includes a gelfoam, such as a cylindrical gel foam. In some embodiments, the tissue device is a gelfoam as provide by Pfizer of New York City, New York. Nevertheless, in some embodiments, the tissue device is coated in gelatin. For instance, in some embodiments, the tissue is coated with about 2% gelatin, about 1.5% gelatin, about 1% gelatin, about 0.5% gelatin, about 0.35% gelatin, about 0.2% gelatin, about 0.1% gelatin, or about 0.05% gelatin.

Accordingly, in some embodiments, the liquid-tight case is utilized for flowing a culture medium through the system. In some embodiments, a plurality of cells is cultured in two dimensions within a respective tissue device (e.g., cultured on a membrane of an insert of a tissue device). In some embodiments, a plurality of cells is cultured in three dimensions within a respective tissue device (e.g., the plurality of cells is embedded within a tissue device and/or included, e.g., suspended, in a hydrogel of the liquid-tight case).

In some embodiments, the tissue device includes a length of about 31 millimeters (mm) (e.g., y-axis length), a width of about 11.4 mm (e.g., x-axis length), and a of about height of 11 mm (e.g., z-axis depth).

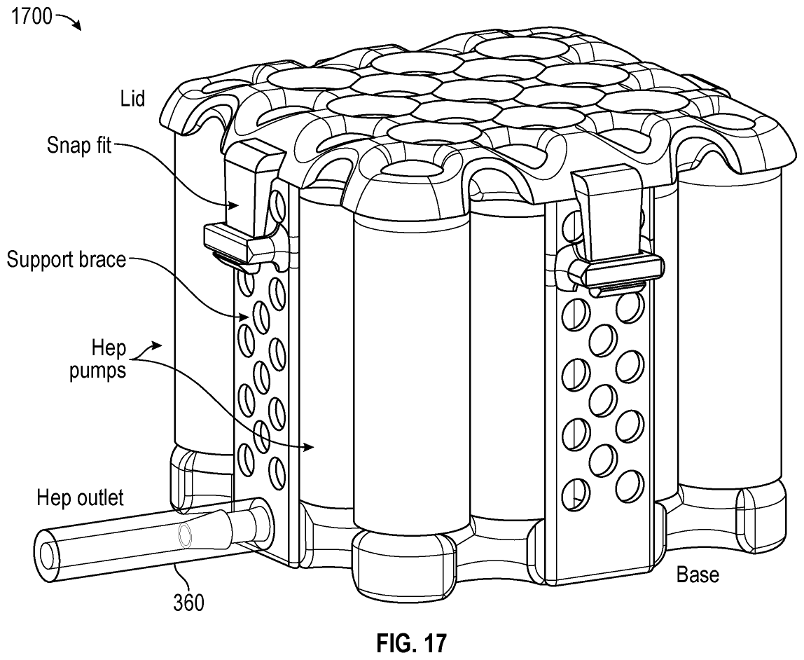

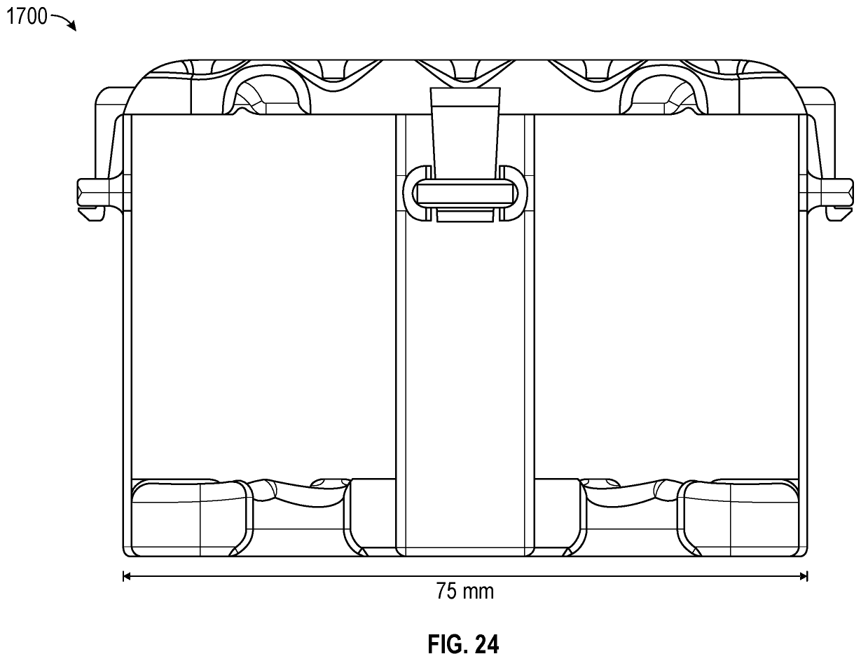

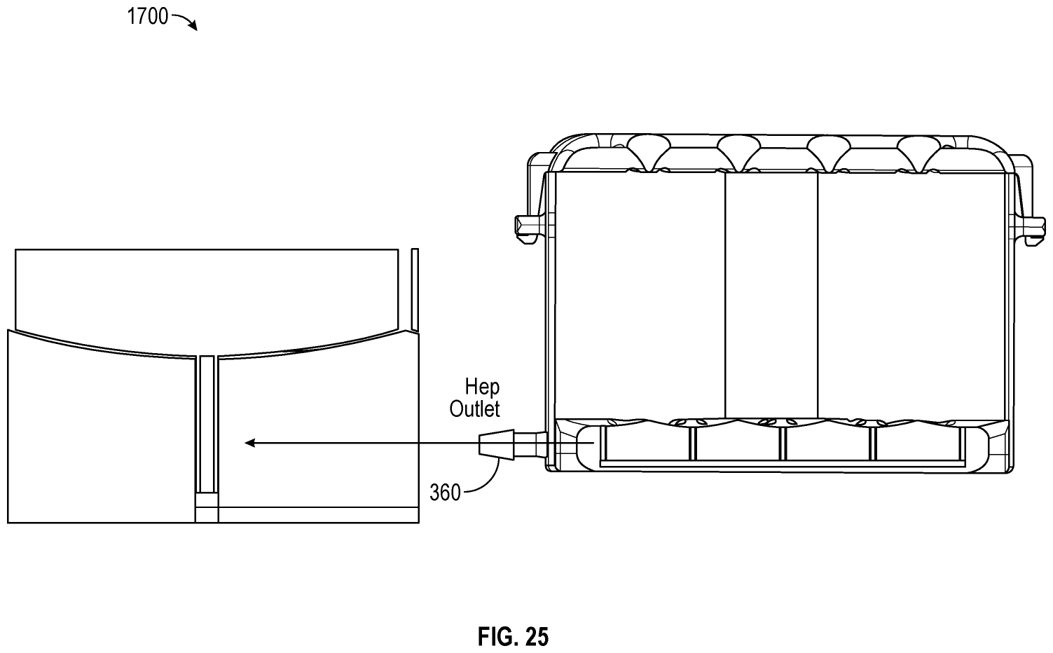

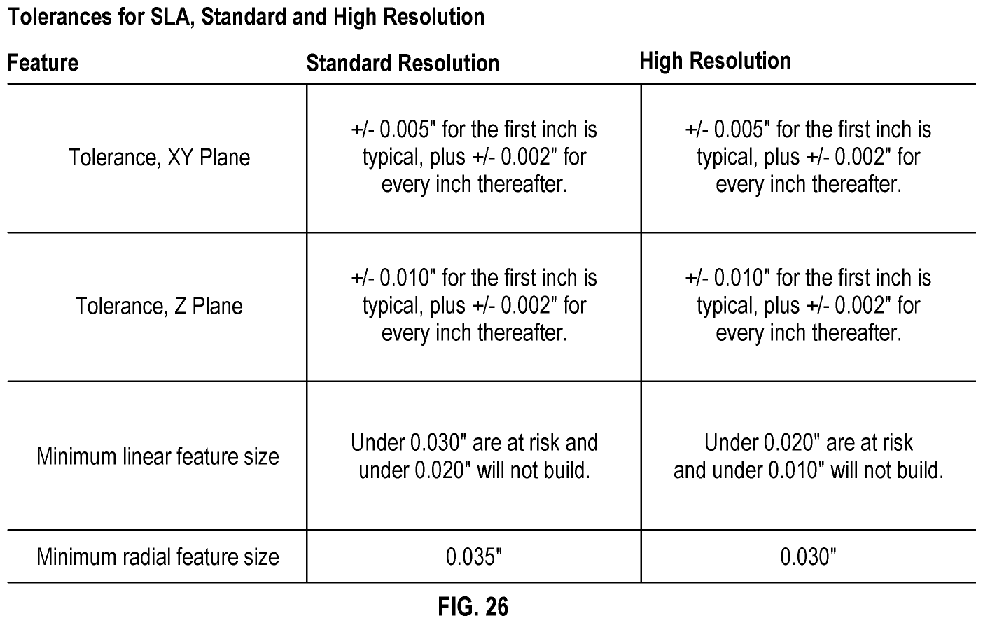

Furthermore, referring FIGS. 17 through 27, in some embodiments, the present invention provides an apparatus for controlling a flow through a liquid-tight case and/or tissue device. In some embodiments, the apparatus includes, or is in electronic communication with, one or more processors, memory coupled to the one or more processors, and a controller coupled to the memory and the one or more processors.

In some embodiments, the flowing of medium within the system is conducted at a flowrate of about 150 microliters per minute (IL/min), about 200 μL/min, about 250 μL/min, about 300 μL/min, about 350 μL/min, about 400 μL/min, about 450 μL/min, about 500 μL/min, about 550 μL/min, about 600 μL/min, about 650 μL/min, or about 700 μL/min. Generally, the flowing of medium within the system is conducted at a flowrate that mimics the flow of fluid within an organ, such that adequate nutrients and waste removal is provided to the cells of the system without damaging the cells.

In some embodiments, the apparatus includes a pump configured to promote a flow of a fluid through the a liquid-tight case and/or tissue device, such as based on one or more instructions provided by the controller.

In some embodiments, the systems and methods of the present invention provide an apparatus, as known as a heparin delivery system (HDS), that includes a holder for up toa a plurality of pumps (e.g., 4 pumps, 8 pumps, 12 pumps, 16 heparin pumps, 20 pumps, 25 pumps, etc.), a collector manifold, an outlet tubing connector, or a combination thereof. In some embodiments, the holder includes a base with manifold and snap lid to secure the pumps.

In some embodiments, manufacture of the systems and apparatus of the present invention uses higher resolution, which may be needed for the manifold channels and holes that mate with the pump needles. However, the present invention is not limited thereto.

In some embodiments, the apparatus is configured to be as light (e.g., low mass) as possible yet configured to make sure the apparatus is sufficiently rigid.

In some embodiments, the systems and apparatus of the present invention is configured to allow for needles to fit into the mating holes of the apparatus.

While the tissue device is illustrated to be of a block, it should be noted that this is by way of simplification of the example. The tissue device has any suitable shape, size and structure. For instance, in some embodiments, the tissue device is selected from a cylinder, sphere, dome, or other shape. Exemplary devices have a dimension that is between about 5 mm and 10 mm, between 10 mm and 20 mm, between 20 mm and 30 mm, between 30 and 40 mm, greater than 40 mm, greater than 50 mm, greater than 60 mm, greater than 70 mm, greater than mm, greater than 90 mm, greater than 100 mm or more. In some embodiments, the tissue device has dimensions of a first dimension about 70 mm, a second dimension of about 50 mm, and a third dimension of about 35 mm.

An exemplary tissue device generally includes one or more channel networks, such as channel network 112. It should be noted that the tissue device can include one, two, three, four or any suitable number of channel networks. It should also be noted that each channel network can include one, two, three, four, five, six, or more than six levels of branching and each branching can be but do not have to be bifurcated. It should be further noted that the channel network(s) can be structured the same as, similar to, or differently from that in the illustrated embodiment. For instance, in some embodiments, the tissue device includes two or more networks, with a first channel network in fluidic communication with a second channel network. The first channel network is configured to receive, through the liquid-tight case, a first plurality of viable cells, and the second channel network is configured to receive, through the liquid-tight case, a second plurality of viable cells. In some embodiments, the tissue device includes a first channel network in fluidic communication with a third channel network, in which a second channel network is formed interposing between the first channel network and the second channel network (e.g., the first channel network bypasses the second channel network, etc.).

In some embodiments, the tissue device is formed using a positive mold, thereby forming a void in between a first channel network and a second channel network. In some embodiments, the forming forms the tissue device with a negative mold, thereby forming a void in between the first channel network and the second channel network of the tissue device. However, the present disclosure is not limited thereto.

In some embodiments, the tissue device is a biomimetic network, a biomimetic structure, a biomimetic device, a vascular network device, an implantable living device or the like. For instance, in some embodiments, the tissue device is a device stimulates an arterial blood flow therethrough. For instance, in some embodiments, the tissue device is configured to satisfy Murray's Law, which is a tool in optimizing a diameter of branching channels of the tissue device. For instance, each preceding channel before a smallest diameter channel of the tissue device has a diameter that is increased by a predetermined factor derived from Murray's Law, which is based on biological observation of a ratio of inflow and outflow diameters. Murray's Law determines that Dₒ³=Σᵢ₌₁ⁿ Dᵢ³, where Dₒ, is a diameter of an upstream parent channel, n is a number of downstream child channels, and Dᵢ is a diameter of an i^{th} child channel. However, the present disclosure is not limited thereto.

In some embodiments, the tissue device is a liver device. In some embodiments, the tissue device is the same as or similar to a biomimetic network, biomimetic structure, biomimetic device, vascular network device, or implantable living device disclosed in any of U.S. Patent Publication No.: 2015/0366651, U.S. Patent Publication No.: 2019/0358367, U.S. Patent Publication No.: 2018/0236134, and U.S. Patent Publication No.: 2021/0071145, each of which is incorporated by reference in its entirety for all purposes.

Referring to FIG. 17, in some embodiments, the apparatus includes a holder for up to 16 heparin pumps, a collector manifold, and outlet tubing connector. The holder includes a base housing the manifold and snap lid to secure the pumps. In some embodiments, the outlet connects to ⅛ inch ID tubing with 1/16 inch wall thickness (so ¼ of an inch outer diameter (OD)). In FIG. 17, the apparatus is illustrated including 16 pumps in the plurality of pumps. However, the present disclosure is not limited thereto.

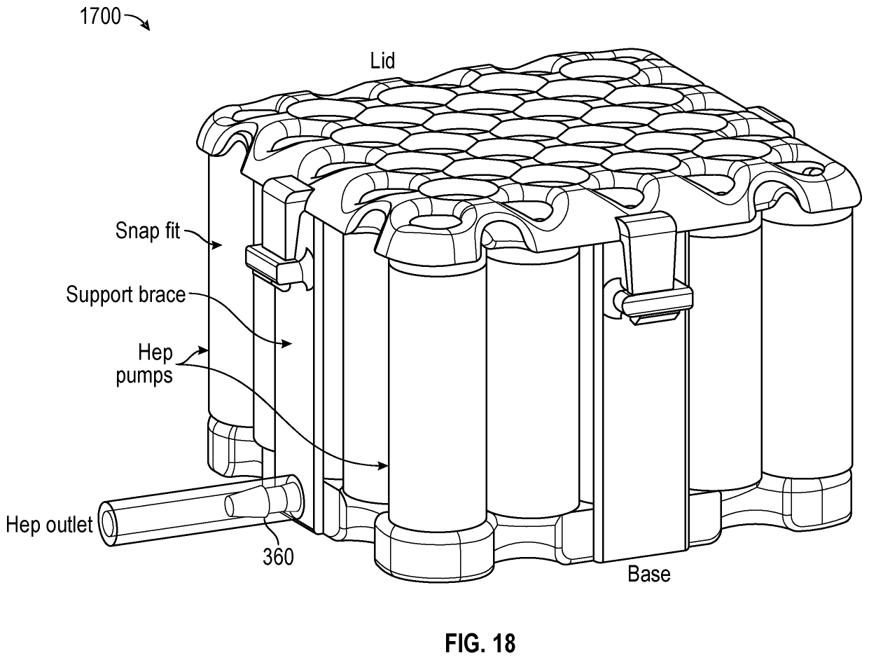

Referring to FIG. 18, in some embodiments, the apparatus satisfies a threshold rigidity, a threshold needle fit, a threshold snap fit, or a combination thereof. For instance, in some embodiments, the threshold snap fit considers if the apparatus couplings are tight, satisfactory, or loose. In some embodiments, the apparatus satisfies a threshold ID of holes for metal needles (e.g., a dimensional threshold), a curvature and tolerance of surfaces threshold that seat the plurality of pumps, a base and/or lid rigidity threshold, a diameter of holes threshold, a rigidity of outlet threshold, a rigidity of side brace supports threshold, a thickness threshold, a circumferential bands (e.g., for additional support and protection of pumps) threshold, or a combination thereof. In FIG. 18, the apparatus is illustrated including 25 pumps in the plurality of pumps.

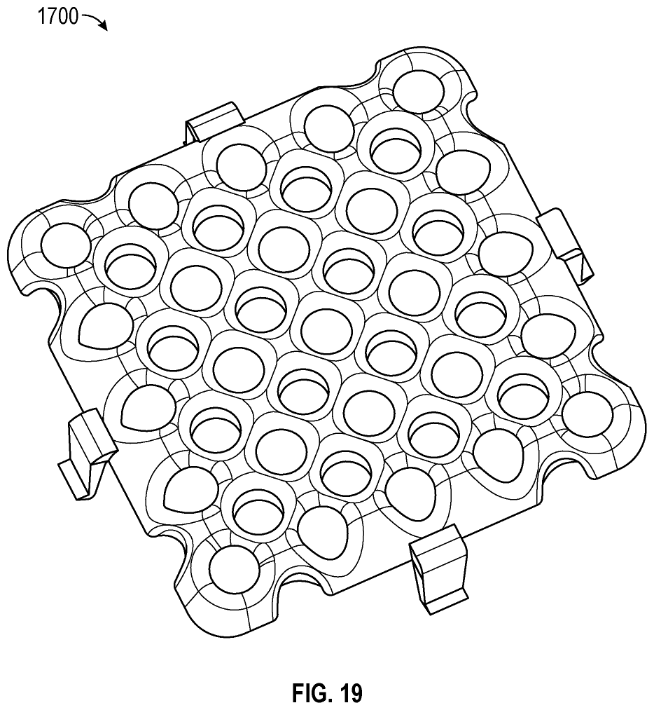

Referring to FIG. 19, in some embodiments, the apparatus includes a first portion (e.g., a lid portion) that is configured to couple the to a second portion (e.g., a holder base portion) of the apparatus. In some embodiments, the first portion is configured to snap-fit couple to the second portion, such as through a cantilever snap fit closure mechanism. In some embodiments, a respective cantilever snap fit closure mechanism is disposed at two or more sides of the first portion of the apparatus.

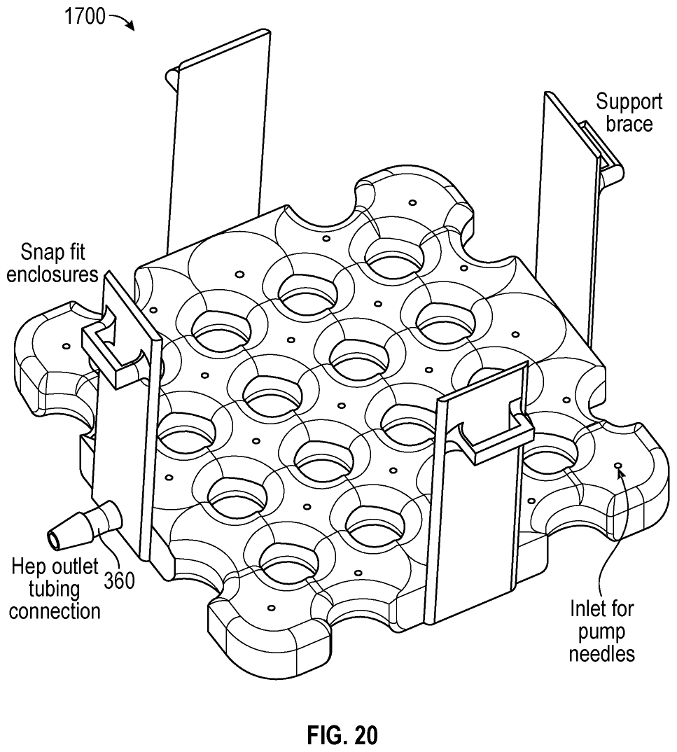

Referring to FIG. 20, in some embodiments, the apparatus includes the second portion that is configured to receive the first portion of the apparatus. In some embodiments, the second portion is configured as a base. In some embodiments, the second portion includes one or more snap fit connections configured to receive a corresponding snap fit mechanism of the first portion of the apparatus. In some embodiments, the second portion includes one or more inlets for the one or more heparin pump needles. In some embodiments, the second portion includes curved surfaces for seating pump surfaces. In some embodiments, the second portion includes a heparin outlet tubing connection.

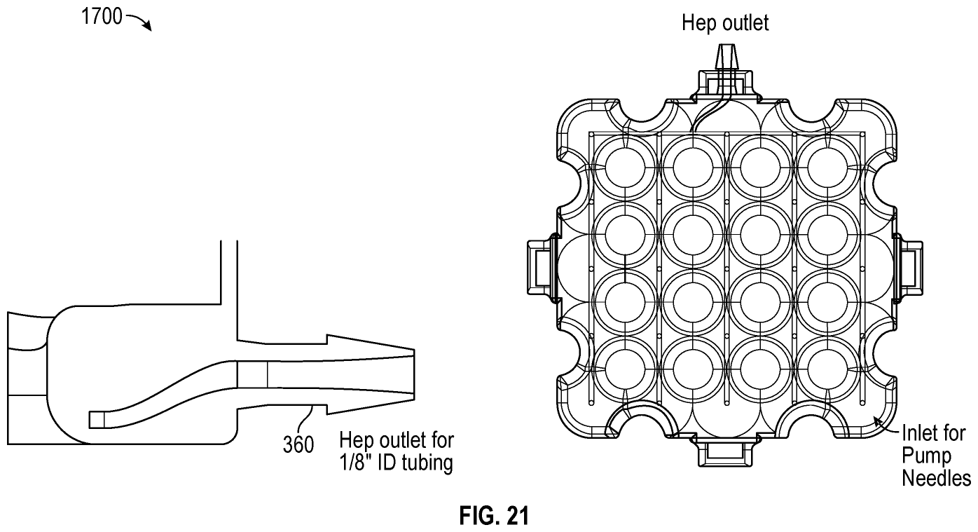

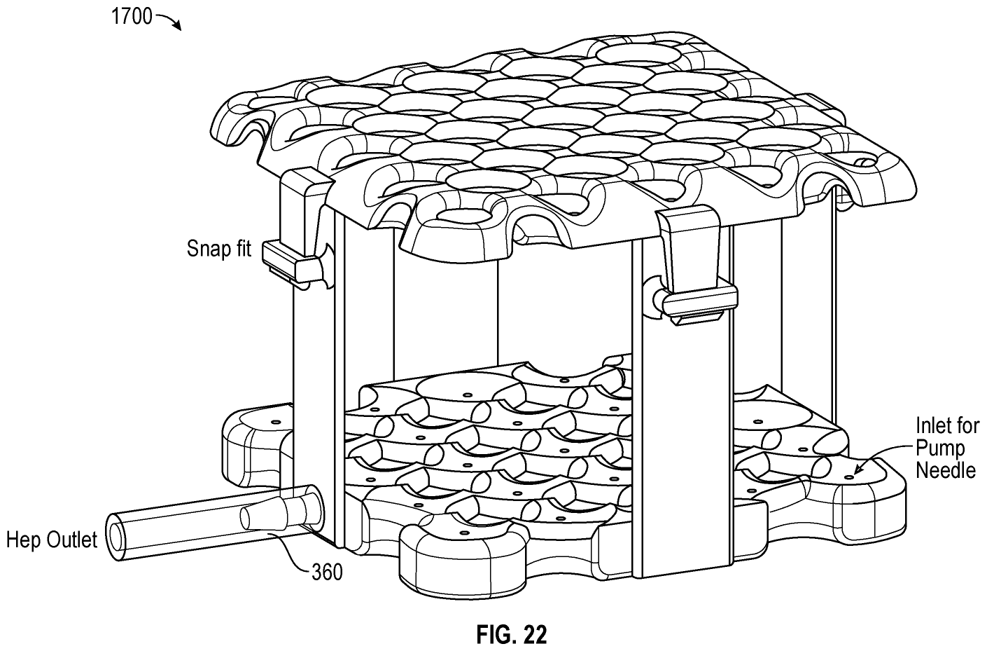

Referring to FIGS. 21 and 22, in some embodiments, the apparatus includes a manifold. In some embodiments, the manifold is a component of the second portion. For instance, in some embodiments, the manifold and the second portion form a three-dimensional monolithic device. In some embodiments, the manifold is a collector coupled to the plurality of pumps. In some embodiments, the manifold is housed in the second portion. In some embodiments, the manifold includes a plurality of holes for mating with pump needles and ends at an outlet tubing connection. In some embodiments, the manifold includes 1 millimeter (mm) square collector channels are 0.5 mm below the needle tips. In some embodiments, the outlet tubing connection is for ⅛ of an inch in internal diameter (ID) tubing. In some embodiments, each pump is configured to provide uniform flow rate of a medium (e.g., provide a constant pressure gradient). In some embodiments, each pump is in fluidic communication with at least one pump in the plurality of pumps. However, the present disclosure in not limited thereto. In some embodiments, a respective pump is configured to provide a flow rate to a liquid-tight case and/or a tissue device at a rate of about 100 milliliter (mL) per hour, about 300 mL per hour, about 500 mL per hour, about 700 mL per hour, about 900 mL per hour, about 1.5 L per hour, about 2 L per hour, about 5 L per hour, or about 30 L per hour. In some embodiments, a respective pump is configured to provide a flow rate to a liquid-tight case and/or a tissue device at a rate of at least about 100 milliliter (mL) per hour, at least about 300 mL per hour, at least about 500 mL per hour, at least about 700 mL per hour, at least about 900 mL per hour, at least about 1.5 L per hour, at least about 2 L per hour, at least about 5 L per hour, or at least about 30 L per hour. In some embodiments, a respective pump is configured to provide a flow rate to a liquid-tight case and/or a tissue device at a rate of at most about 100 milliliter (mL) per hour, at most about 300 mL per hour, at most about 500 mL per hour, at most about 700 mL per hour, at most about 900 mL per hour, at most about 1.5 L per hour, at most about 2 L per hour, at most about 5 L per hour, or at most about 30 L per hour.

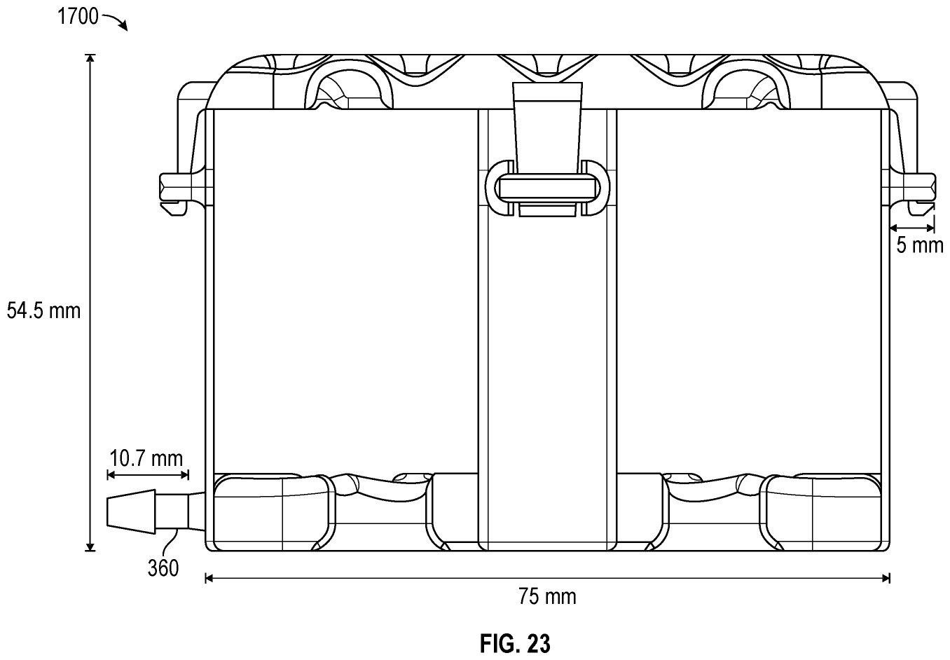

Referring to FIGS. 23 through 26, in some embodiments, the apparatus is configured to satisfy one or more dimensional thresholds, such as a tolerance of an XY plane, a tolerance of a Z plane, a minimum linear feature size, a minimum radial feature size, or a combination thereof.

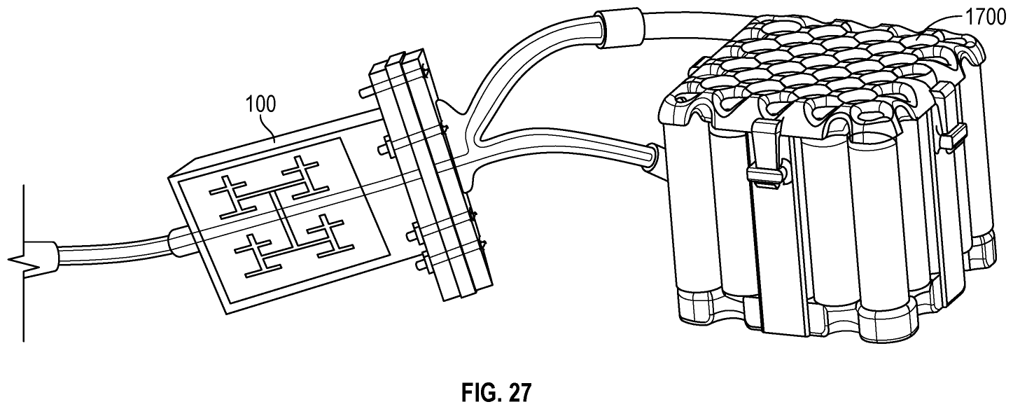

Referring to FIG. 27, a system 100 is provided that includes a tissue device, such as tissue device 110, and a liquid-tight case, such as liquid-tight case 120, and an apparatus for controlling a flow through the liquid-tight case and/or the tissue device.

It will be appreciated by those skilled in the art that changes could be made to the exemplary embodiments shown and described above without departing from the broad inventive concepts thereof. It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways.

Specific features of the exemplary embodiments may or may not be part of the claimed invention and various features of the disclosed embodiments may be combined. Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Unless specifically set forth herein, the terms “a”, “an” and “the” are not limited to one element but instead should be read as meaning “at least one”. The words “right”, “left”, “top”, “bottom”, “inner” and “outer” designate directions in the drawings to which reference is made.

### Example I—Culturing of Hepatocyte Cells

A liquid tight case of a system includes a plurality of hepatocyte cells accommodated in a tissue device. Upon introduction of a medium, the hepatocyte cells will produce a blood effluent and a bile effluent. Accordingly, pumps are coupled to the liquid tight case such that an inlet of a channel of the tissue device is configured to receive the blood effluent while the outlet of the channel is configured to receive the bile effluent. However, the present disclosure is not limited thereto. In some embodiments, this configuration allows for various experiments and determinations to be conducted on each respective effluent (e.g., determine if the respective effluent will kill or not kill a subsequent cell).

Accordingly, the systems, methods, and apparatuses of the present disclosure provide a means for directing flow within one or more liquid-tight cases. In some embodiments, these liquid-tight cases are utilized to simulate cell culture in vivo. In some embodiments, the liquid-tight case is capable of culturing cells with a volume of approximately 1,000 mm³.

The foregoing descriptions of specific exemplary embodiments of the present invention have been presented for purposes of illustration and description. They are not intended to be exhaustive or to limit the invention to the precise forms disclosed, and obviously many modifications and variations are possible in light of the above teachings. The exemplary embodiments were chosen and described in order to explain certain principles of the invention and their practical application, to thereby enable others skilled in the art to make and utilize various exemplary embodiments of the present invention, as well as various alternatives and modifications thereof. It is intended that the scope of the invention be defined by the Claims appended hereto and their equivalents.

1. A liquid-tight case comprising:
a housing configured to enclose a tissue device within, wherein the tissue device comprises a first channel network and a second channel network in fluidic communication with the first channel network;
a first inlet port or coupled with the housing, and configured to be in fluidic communication with the first channel network of the tissue device;
a second inlet port coupled with the housing, and configured to be in fluidic communication with the second channel network of the tissue device; and
at least one outlet port coupled with the housing.

2. A liquid-tight case comprising:
a housing configured to enclose a tissue device within;
a first inlet port coupled with the housing, and configured to receive a first plurality of viable cells and pass the first plurality of viable cells to the tissue device;
a second inlet port coupled with the housing, and configured to receive a second plurality of viable cells and pass the second plurality of viable cells to the tissue device; and
at least one outlet port coupled with the housing.

3. The liquid-tight case of claim 2, wherein the tissue device comprises:
a first channel network in fluidic communication with the first inlet port of the liquid-tight case to receive the first plurality of viable cells; and
a second channel network in fluidic communication with the second inlet port of the liquid-tight case to receive the second plurality of viable cells,
wherein the first and second channel networks are in fluidic communication with each other.

4. The liquid-tight case of claim 2, wherein the tissue device is a device stimulates an arterial blood flow.

5. The liquid-tight case of claim 4, wherein the tissue device is a liver device.

6. The liquid-tight case of claim 2, wherein the at least one outlet port comprises a first outlet port in fluidic communication with the first inlet port through the tissue device.

7. The liquid-tight case of claim 2, wherein the housing comprises:
a base formed with a hollow space configured to house the tissue device;
a lid coupled with the base to enclose the tissue device within the hollow space; and
a seal disposed between the base and the lid to enhance sealing of the liquid-tight case.

8. The liquid-tight case of claim 7, wherein the lid is coupled with the bases by one or more fasteners, snap-fitting, press-fitting, one or more adhesives, tape, or a combination thereof.

9. The liquid-tight case of claim 7, wherein the seal is made of an elastomeric material.

10. The liquid-tight case of claim 7, wherein the seal is an O-ring.

11. The liquid-tight case of claim 10, wherein the base comprises a rim and a groove formed along the rim to accommodate the seal.

12. The liquid-tight case of claim 7, wherein the first inlet port, second inlet port and at least one outlet port are coupled with the lid or base.

13. The liquid-tight case of claim 12, wherein the first inlet port, the second inlet port, or each of the first and second inlet ports comprises a barbed outer connector coupled with an exterior side of the lid or base to facilitate tubing.

14. The liquid-tight case of claim 12, wherein:
the at least one outlet port comprises a first outlet port; and
the base of the housing is monolithically formed with a sheath configured to allow tubing of the first outlet port to pass through and be connected directly to the tissue device.

15. The liquid-tight case of claim 12, wherein the first and second inlet ports are coupled with a first portion of the base and the at least one outlet port is coupled with a second portion of the base.

16. The liquid-tight case of claim 15, wherein each of the first and second inlet ports comprises a barbed outer connector formed at an exterior side of the first portion of the base to facilitate tubing, and wherein a barbed inner connector is formed at an interior side of the first portion of the base to facilitate tubing and direct connection to the tissue device.

17. The liquid-tight case of claim 15, wherein the first and second portions of the base are opposite to each other.

18. The liquid-tight case of claim 12, wherein the first and second inlet ports are coupled with the lid.

19. The liquid-tight case of claim 18, wherein each of the first and second inlet ports comprises a barbed outer connector coupled with an exterior side of the lid to facilitate tubing, and wherein a barbed inner connector is coupled with an interior side of the lid to facilitate tubing and direct connection to the tissue device.

20. A system comprising:
the liquid-tight case of claim 1; and
the tissue device enclosed within the liquid-tight case and in fluidic communication with the first inlet port, the second inlet port and the at least one outlet port.
